(19) United States
(12) Patent Application Publication
(10) Pub. No.: US 2026/0179719 A1
(43) Pub. Date: Jun. 25, 2026

(54) MULTIMODAL COMPOUND GENERATION SYSTEM, METHOD AND MULTIMODAL CUSTOMIZED COMPOUND GENERATION SYSTEM

(71) Applicant: Industrial Technology Research Institute, Hsinchu (TW)

(72) Inventors: Hsiang-Wei Hu, Tainan City (TW); Shu-Fan Wang, Hsinchu County (TW); Cheng-Kuo Lai, Taichung City (TW); Chun-Hung Lien, Taipei City (TW); Kai-Cheng Hsu, Chiayi County (TW)

(73) Assignee: Industrial Technology Research Institute, Hsinchu (TW)

(21) Appl. No.: 19/263,543

(22) Filed: Jul. 9, 2025

(30) Foreign Application Priority Data
Dec. 25, 2024 (TW) 113150565

(51) Int. Cl. G16B 15/00; G16B 30/00; G16B 40/20

(52) U.S. Cl. CPC G16B 15/00; G16B 30/00; G16B 40/20

(57) ABSTRACT

In the disclosure, compounds may be generated by taking into account the characteristics of target proteins in different dimensions via a multimodal compound generation system and method. The compounds have stronger binding characteristics with the target proteins than molecules produced by previous methods taking into account a single dimension. In addition, modified compounds may be produced from known compound molecules and used to train the neural network model to improve the quality of future compound production. The disclosure also provides a multimodal customized compound generation system for the user to input expected parameters to produce compounds that meet user expectations.

### CROSS-REFERENCE TO RELATED APPLICATION

This application claims the priority benefit of Taiwan application serial no. 113150565, filed on Dec. 25, 2024. The entirety of the above-mentioned patent application is hereby incorporated by reference herein and made a part of this specification.

### TECHNICAL FIELD

The disclosure relates to a compound generation system, method, and customized compound generation system, and more particularly to a multimodal compound generation system, method, and multimodal customized compound generation system.

### BACKGROUND

In the traditional drug development process, single-modality data, such as genome sequence or protein structure, is usually used to design potential drug molecules. However, such an approach may not fully take into account the complex interactions between the drug and the target protein, resulting in a higher failure rate during the development process. Therefore, how to incorporate the interaction between drugs and target proteins into the parameters of drug development is an important topic.

### SUMMARY

Accordingly, the disclosure provides a multimodal compound generation system, method, and multimodal customized compound generation system integrating genetic data, protein secondary structure, and three-dimensional structure data to form one unified analysis framework, so as to accordingly design desirable compound molecules.

A multimodal compound generation system of the disclosure includes a storage and a processor. The storage stores a plurality of models. The processor is coupled to the storage and configured to: input a plurality of parameters to a neural network model in the plurality of models, wherein there is a first correlation between a first parameter and a second parameter in the plurality of parameters, and there is a second correlation between the second parameter and a third parameter in the plurality of parameters; and obtain information of a first compound via the neural network model, wherein a first characteristic of the first compound is greater than a first threshold value, and a second characteristic of the first compound is greater than a second threshold value.

In an embodiment of the disclosure, the processor of the multimodal compound generation system is further configured to: execute a heterogeneous data fusion algorithm to pre-process the plurality of parameters to obtain a plurality of pre-processing parameters, wherein each of the plurality of pre-processing parameters has a same format.

In an embodiment of the disclosure, the plurality of parameters of the multimodal compound generation system include a one-dimensional structure, a two-dimensional structure, and a three-dimensional structure.

In an embodiment of the disclosure, the processor of the multimodal compound generation system is further configured to: execute a feature extraction model in the plurality of models to extract a plurality of features of a target compound, wherein the plurality of features are related to the plurality of parameters.

In an embodiment of the disclosure, the storage of the multimodal compound generation system further stores a plurality of modules, and the processor is further configured to: execute a prediction module in the plurality of modules to predict an activity and a stability of the first compound.

In an embodiment of the disclosure, the processor of the multimodal compound generation system is further configured to: execute a training module in the plurality of modules to train the neural network model according to information of the first compound to obtain a trained neural network model; and input the plurality of parameters into the trained neural network model to obtain information of a second compound.

In an embodiment of the disclosure, the processor of the multimodal compound generation system is further configured to: determine information of the first compound meets a first expectation.

In an embodiment of the disclosure, in the multimodal compound generation system, the first parameter is a polypeptide sequence, the second parameter is a secondary structure of a protein, and the third parameter is a three-dimensional structure of a protein.

A multimodal compound generation method of the disclosure includes: inputting a plurality of parameters to a neural network model, wherein there is a first correlation between a first parameter and a second parameter in the plurality of parameters, and there is a second correlation between the second parameter and a third parameter in the plurality of parameters; and obtaining information of a first compound via the neural network model, wherein a first characteristic of the first compound is greater than a first threshold value, and a second characteristic of the first compound is greater than a second threshold value.

In an embodiment of the disclosure, the multimodal compound generation method further includes: executing a heterogeneous data fusion algorithm to pre-process the plurality of parameters to obtain a plurality of pre-processing parameters, wherein each of the plurality of pre-processing parameters has a same format.

In an embodiment of the disclosure, the plurality of parameters of the multimodal compound generation method include a one-dimensional structure, a two-dimensional structure, and a three-dimensional structure.

In an embodiment of the disclosure, the multimodal compound generation further includes: executing a feature extraction model to extract a plurality of features of a target compound, wherein the plurality of features are related to the plurality of parameters.

In an embodiment of the disclosure, the multimodal compound generation method further includes: executing a prediction module to predict an activity and a stability of the first compound.

In an embodiment of the disclosure, the multimodal compound generation method further includes: executing a training module to train the neural network model according to information of the first compound to obtain a trained neural network model; and inputting the plurality of parameters into the trained neural network model to obtain information of a second compound.

In an embodiment of the disclosure, the multimodal compound generation method further includes: determining information of the first compound meets a first expectation.

In an embodiment of the disclosure, the first parameter of the multimodal compound generation method is a polypeptide sequence, the second parameter is a secondary structure of a protein, and the third parameter is a three-dimensional structure of a protein.

A customized multimodal compound generation system of the disclosure includes a storage and a processor. The storage stores a plurality of models. The processor is coupled to the storage and configured to: obtain information of a first compound; obtain a plurality of basic parameters according to the first compound; and execute the above method to obtain information of a second compound.

In an embodiment of the disclosure, the multimodal customized compound generation system further includes a user interface coupled to the processor, and the processor is further configured to: obtain a first threshold value and a second threshold value via the user interface.

In an embodiment of the disclosure, the storage of the multimodal customized compound generation system further stores a plurality of modules, wherein the processor is further configured to: execute a quality monitoring module in the plurality of modules to determine whether the information of the second compound meets a quality threshold value.

In an embodiment of the disclosure, the processor of the multimodal customized compound generation system is further configured to: execute a performance monitoring module in the plurality of modules to monitor a training performance of the neural network model.

Based on the above, in the disclosure, compounds may be generated by taking into account the characteristics of target proteins in different dimensions via a multimodal compound generation system and method. The compounds have stronger binding characteristics with the target proteins than molecules produced by previous methods taking into account a single dimension. In addition, modified compounds may be produced from known compound molecules and used to train the neural network model to improve the quality of future compound production. The disclosure also provides a multimodal customized compound generation system for the user to input expected parameters to produce compounds that meet the user's expectations.

### BRIEF DESCRIPTION OF THE DRAWINGS

FIG. 1 is a schematic diagram of a multimodal compound generation system of the disclosure.

FIG. 2 is a schematic diagram of an implementation flow of a multimodal compound generation system of the disclosure.

FIG. 3 is a detailed flowchart of the disclosure.

FIG. 4 is a schematic diagram of the implementation process of a multimodal customized compound generation system of the disclosure.

### DETAILED DESCRIPTION OF DISCLOSED EMBODIMENTS

Reference will now be made in detail to exemplary embodiments of the disclosure, examples of which are illustrated in the accompanying drawings. Terms such as “first” and “second” mentioned throughout the specification (including the claims) of the present application are used to name elements or to distinguish between different embodiments or scopes, and are not used to limit the upper bound or the lower bound of the number of elements, nor used to limit the sequence of elements. In addition, wherever possible, elements/members using the same reference numerals in the drawings and embodiments denote the same or similar parts.

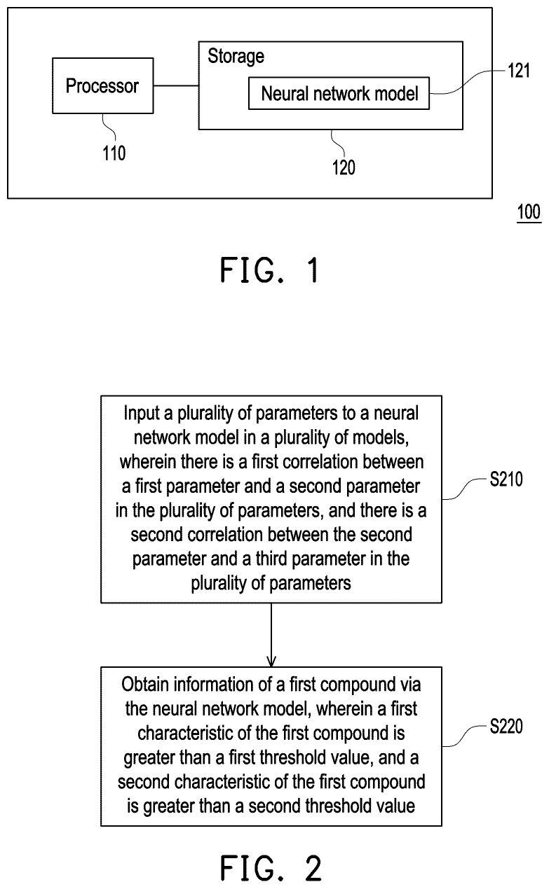

FIG. 1 is a schematic diagram of a multimodal compound generation system provided by the disclosure. A multimodal compound generation system 100 may include a processor 110 and a storage 120.

In an embodiment of the disclosure, the processor is, for example, a central processing unit (CPU), or other programmable general-purpose or special-purpose micro control unit (MCU), microprocessor, digital signal processor (DSP), programmable controller, application-specific integrated circuit (ASIC), graphics processing unit (GPU), image signal processor (ISP), image processing unit (IPU), arithmetic logic unit (ALU), complex programmable logic device (CPLD), field-programmable gate array (FPGA), or other similar elements, or a combination of the above elements. In the multimodal compound generation system 100, the processor 110 may be coupled to the storage 120, and the processor 110 may execute each module and each model stored in the storage 120.

The storage 120 is, for example, any type of fixed or removable random-access memory (RAM), read-only memory (ROM), flash memory, hard-disk drive (HDD), solid-state drive (SSD), or similar elements, or a combination of the above elements, and is used to store a plurality of modules, a plurality of models, or various applications that may be executed by the processor 110. In the present embodiment, the storage 120 may store at least a neural network model 121.

Please refer to FIG. 2. FIG. 2 is a schematic diagram of the implementation flow of a multimodal compound generation system of the disclosure that may be implemented by the processor 110. In step S210, the processor 110 may input a plurality of parameters to a neural network model in the plurality of models, wherein there is a first correlation between a first parameter and a second parameter in the plurality of parameters, and there is a second correlation between the second parameter and a third parameter in the plurality of parameters. In step S220, the processor 110 may obtain information of a first compound via the neural network model, wherein a first characteristic of the first compound is greater than a first threshold value, and a second characteristic of the first compound is greater than a second threshold value.

Specifically, the structure of a molecule includes a one-dimensional atomic arrangement, a secondary structure formed by hydrogen bonds, and a folded three-dimensional structure. In the past, drug development or compound synthesis took into account a single dimension and ignored the impact of other dimensions. As a result, the designed compound molecules have weak binding affinity with the target protein (such as a mutant protein causing lesions).

In the following embodiments, the design of compounds (drugs) for amyotrophic lateral sclerosis (ALS) is taken as an example for detailed description. Abnormal aggregation of TDP-43 (TAR DNA-binding protein 43) is a common pathological feature of ALS. The abnormal aggregation of this protein leads to degenerative changes in neurons.

In an embodiment of the disclosure, TDP-43 is first disassembled to obtain the one-dimensional amino acid sequence, the secondary hydrogen bond structure, and the three-dimensional folding structure of TDP-43. The above may include obtaining the base pair sequence (gene sequence feature) regulating TDP-43 via the data of the gene library, and then obtaining the transcribed and translated amino acid sequence. Moreover, since the amino acid sequence is determined, hydrogen bonds in the amino acids may be formed accordingly, resulting in specific folding patterns, including a-helix and β-sheet. In addition, the three-dimensional structure of the TDP-43 protein may be obtained via X-ray crystallography or cryo-electron microscopy data. In this way, in the disclosure, key features may be extracted from the gene sequence features, the secondary hydrogen bond structure, and the three-dimensional structure of the TDP-43 protein. Alternatively, in the disclosure, the multi-dimensional parameters may be processed as heterogeneous data to avoid compressing any dimensional parameter and causing information loss, so as to generate corresponding compounds. In an embodiment of the disclosure, the one-dimensional amino acid sequence (peptide sequence), the secondary hydrogen bonding structure, and the three-dimensional folding structure of TDP-43 all belong to one mode.

In an embodiment of the disclosure, the processor 110 may execute a heterogeneous data fusion algorithm to pre-process parameters of a plurality of different units to obtain a plurality of pre-processing parameters, so that the units or the formats of the pre-processing parameters are all the same. For example, as may be seen from the previous paragraph, in order to integrate parameters including one-dimensional amino acid sequences of different modes, secondary hydrogen bond structures, and three-dimensional folding structures, the processor 110 may integrate the parameters in different units via a heterogeneous data fusion algorithm to convert each parameter into the same unit. The one-dimensional amino acid sequence is a one-dimensional structure, the secondary hydrogen bond structure is a two-dimensional structure, and the three-dimensional folded structure is a three-dimensional structure.

Specifically, in an embodiment of the disclosure, since the one-dimensional amino acid sequence of a protein affects the secondary structure of the protein, the secondary structure of the protein in turn affects the three-dimensional structure of the protein. Therefore, there is at least a first correlation between the one-dimensional amino acid sequence and the secondary structure, and there is at least a second correlation between the secondary structure and the three-dimensional structure. Therefore, in an embodiment of the disclosure, at least the related features of the one-dimensional amino acid sequence and the secondary structure may be extracted and represented in the same format, and the related features of the secondary structure and the three-dimensional structure may be extracted and represented in the same format. Lastly, after pre-processing, the format of each modality may be organized into the same representation.

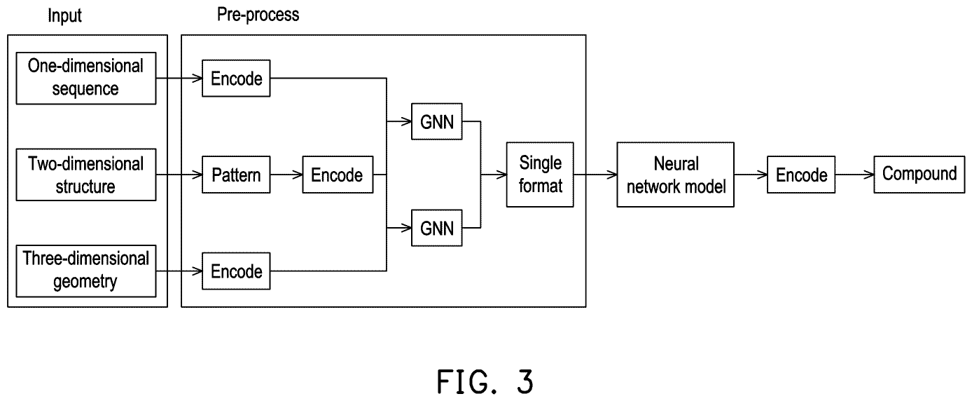

Please refer to FIG. 3. FIG. 3 is a detailed flowchart of the disclosure. After the compound parameters of the one-dimensional sequence, the two-dimensional structure, and the three-dimensional geometry of TDP-43 are input, the processor 110 may pre-process each compound parameter to obtain a single format. The processor 110 may first encode the one-dimensional sequence and obtain features, molecular fingerprints, and SMILES-transformers via the simplified molecular input line entry specification (SMILES) to obtain atomic and molecular feature embedding. The processor 110 performs patterning on the two-dimensional structure and then performs encoding. The processor 110 further parses the three-dimensional geometric structure to obtain a plurality of information.

Please continue to refer to FIG. 3, after the processor 110 encodes the compound parameters of the one-dimensional sequence, the two-dimensional structure, and the three-dimensional geometry respectively, the processor 110 fuses the information of the first code of the one-dimensional atomic and molecular features and the second code of the two-dimensional structure via a neural network algorithm to obtain a first related format. The processor 110 further fuses the plurality of messages of the second code of the two-dimensional structure and the third code of the three-dimensional geometric structure to obtain a second related format. In an embodiment of the disclosure, the processor 110 may process the above process via a graph neural network (GNN). In other embodiments of the disclosure, the processor 110 may also perform the above process via other neural network algorithms.

Continuing from the above paragraph, the processor 110 further integrates the first related format and the second related format into a single format. In addition, this single format may be further decoded to generate compounds after being input into the neural network model. In an embodiment of the disclosure, the neural network model may be, for example, a transformer.

Specifically, the processor 110 may extract features via the following three chemical molecular fingerprint methods. The processor 110 executes MACCS and extracts the structural and functional features (e.g., rings, chains, functional groups, etc.) of the molecule and represents each structural and functional feature with a 166-bit binary code, and lastly combines the above with a classifier to evaluate the activity of the compound. Via PubChem, the processor 110 may also set the corresponding binary position to 1 or 0 according to whether a specific substructure exists in the molecule and form an 881-bit binary code, and lastly use the above for rapid comparison and screening of compounds in the compound library. The processor 110 may also identify key features of the interaction between molecules and receptors via ErG. The key features include hydrogen bonds, hydrophobic regions, and the key features are converted into radial distribution functions and integrated into the descriptor vector, and is lastly used for small molecule virtual screening.

Continuing from the previous paragraph, the processor 110 may encode the three-dimensional geometric structure by: task 1: masking atomic features and reconstructing atoms; task 2: masking the 3D coordinates and reconstructing the coordinates, and task 3: masking atoms and 3D coordinates independently and reconstructing the masked portions. In task 1, the processor 110 aims to enhance the ability of the model to extract two-dimensional graphic information. Therefore, the processor 110 masks the chemical features of all atoms, predicts the atomic features using the three-dimensional coordinates to preserve the topology of the atomic structure, and calculates loss using the cross entropy loss function. In task 2, the processor 110 aims to enhance the ability of the model to generate three-dimensional coordinates and extract relevant three-dimensional information. Therefore, the processor 110 masks the 3D coordinates of all atoms and predicts the 3D structure using the atomic features and considers rotation and permutation invariance using a loss function. In task 3, the processor 110 aims to improve the comprehensive information fusion capability of the model. Therefore, the processor 110 masks atoms and structurally reconstructs the task description, that is, masks and reconstructs atoms and structures of the molecular graph, which is an extreme case of combining task one and task two.

In an embodiment of the disclosure, after the GNN processing, the processor 110 may integrate the one-dimensional sequence, the secondary structure, and the three-dimensional structure into graph-based parameters and input the parameters into the neural network model to generate compounds.

In an embodiment of the disclosure, the processor 110 may generate a corresponding compound structure by analyzing the target protein, and the compound may generate binding affinity with the target protein. The processor 110 further executes a prediction module to predict the activity and the stability of the compound. Specifically, the molecular characteristics of the resulting compound include number of atoms (num_atoms), molecular weight (mwt), oil-water partition coefficient (logP), number of hydrogen bond acceptors (HAcceptors), number of hydrogen bond donors (HDonors), topological polar surface area (TPSA), number of rotatable bonds (rotatableBonds), drug-like properties (QED), and synthesizability score (SAscore). In an embodiment of the disclosure, the molecular characteristics of the resulting compound at least meet the above two threshold values, that is, the first characteristic is greater than the first threshold value and the second characteristic is greater than the second threshold value, so that the resulting compound meets the requirements. The first threshold value and the second threshold value may have different values depending on different target proteins.

In addition to the molecular characteristics, there is a certain possibility that the resulting compound is not highly active or is unstable due to analyzing the target protein and correspondingly generating the compound. For example, a compound molecule generated by machine learning contains a one-dimensional sequence, which may be unstable and arbitrarily folded into a three-dimensional structure that may not target the target protein after being generated. In this case, the resulting compound is not a stable protein.

Continuing from the previous paragraph, the structure of the compound produced by machine learning may also be unreasonable. For example, in order to simultaneously target the one-dimensional sequence, the secondary structure, and the three-dimensional structure of a target protein, one compound with a molecular weight much larger than that of the target protein is produced, resulting in the compound being unable to effectively bind to the target protein. The structure of the resulting compound is unreasonable as described above. Therefore, after the processor 110 generates the compound via the machine learning model, the processor 110 may execute the analysis module to determine whether the information of the compound meets expectations. The expectation is, for example, that the molecular weight of the compound is not greater than one-tenth of the target protein. The implementer of the disclosure may adjust the expected items and the corresponding threshold values according to actual applications.

In another embodiment of the disclosure, the processor 110 may improve a known compound so that the improved compound has a stronger binding affinity with the target protein than the original known compound. The processor 110 may execute a feature extraction model to extract a plurality of features of a compound from a known compound. Since the object of the compound is to bind to a target protein, a plurality of features of the compound are associated with the parameters of the one-dimensional sequence, the secondary structure, and the three-dimensional structure of the protein. The processor 110 may train the neural network model using a plurality of features of the known compound to obtain a trained neural network model. In addition, the processor 110 may also obtain the recommended compound recommended by the neural network model, and after the processor 110 determines that the recommended compound meets expectations, the processor 110 retrains the neural network model with the information of the composition and the features of the recommended compound to obtain a trained neural network model. The processor 110 also inputs a plurality of parameters of the target protein into the trained neural network model to obtain information of the second compound. By means of the training method, the neural network model may be optimized, and the processor 110 may obtain the optimized compound from the optimized neural network model.

The disclosure also provides a multimodal compound generation method that may be implemented by the processor 110 of the multimodal compound generation system. The specific implementation is described above and is not described in detail here.

The disclosure also provides a multimodal customized compound generation system including a processor and a storage. In an embodiment of the disclosure, the processor of the multimodal customized compound generation system may first obtain information of a known compound, and the processor obtains a plurality of basic parameters according to the known compound. Next, the processor may obtain a second compound by executing the multimodal compound generation method.

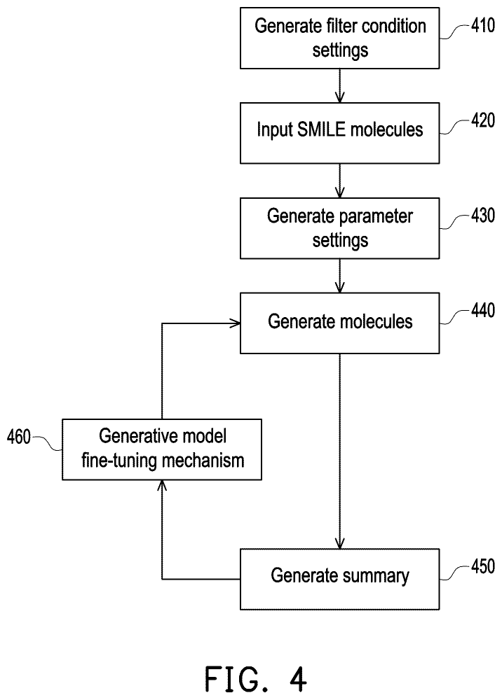

Please refer to FIG. 4. FIG. 4 is a schematic diagram of the implementation process of a multimodal customized compound generation system of the disclosure. In step 410, the processor may generate a filter condition setting. In step 420, the process may receive a SMILE molecule input by a user. In step 430, the processor may generate parameter settings. In step 440, the processor may generate a molecule. In step 450, the processor may generate a summary. In step 460, the processor may generate a model fine-tuning mechanism. As shown in the figure, after step 460, the processor may execute molecule generation again to produce another molecule.

In detail, the object of the multimodal customized compound generation system is to provide the customer with input threshold values of the characteristics of the desired compound. For example, the number of atoms of the compound that the customer wants to obtain needs to be less than 200 and the molecular weight thereof needs to be less than 5000. Customers may input the characteristic threshold values of the molecules they want to obtain therein via the user interface of the multimodal customized compound generation system. The user inputs at least two threshold values of compound characteristics so that the compounds generated by the multimodal customized compound generation system better meet user needs.

More specifically, in step 410, the processor may generate a filter condition setting. For example, the processor may choose to filter out molecules other than ZINC15. The processor may also choose to filter out molecules that are not within the characteristic range. Accordingly, most of the molecules are initially filtered out, thereby increasing the speed at which the neural network model recommends compounds. Next, in step 420, a plurality of molecules may be input. That is, the user may input the SMILE molecule according to their needs, so that the compounds generated by the neural network model include the SMILE molecule. The generation parameter setting in step 430 includes setting the number of input molecules and setting the number of samples. The user may input a string number of molecules from 1 to SMILE. The input SMILE number is, for example, 50. The implementer of the disclosure may determine the number of inputs according to the desired number of molecules and the amount of computable resources. The user may also set the number of samples generated by the neural network model, which may include at least one sample to a maximum of ten samples.

Continuing from the previous paragraph, in step 440, the method of generating compound molecules may adopt a molecular generation model fine-tuned for a specific molecule (the basic framework is MegaMolBART). The model is generated in the form of an API and provides a plurality of generation requirements. In the process of generating compound molecules, the multimodal customized compound generation system may view the structure diagram and the molecular characteristics of the input SMILE molecules via a display interface (user interface), and the display interface may also display the molecular characteristics of all generated molecules at each generation stage. The molecular characteristics include number of atoms (num_atoms), molecular weight (mwt), oil-water partition coefficient (logP), number of hydrogen bond acceptors (HAcceptors), number of hydrogen bond donors (HDonors), topological polar surface area (TPSA), number of rotatable bonds (rotatableBonds), drug-like properties (QED), and synthesizability score (SAscore). Accordingly, the user may ensure that the molecular characteristics meet their needs.

Continuing from the previous paragraph, in step 450, the processor may summarize and display the structure diagrams and the molecular characteristics of the molecules generated in all stages via a display interface. In this way, the user may give molecular evaluations to the compounds recommended by the neural network model using the expert annotation function on the user interface and based on their own experience, molecular structure diagrams, and summaries of molecular characteristics. It should be understood that this evaluation may serve as a training basis for re-optimization of the neural network model. Therefore, in step 460, the fine-tuning mechanism of the neural network model generation model may collect all molecules with high synthetic performance evaluated by experts as molecular data for the next fine-tuning. In addition, the processor may also use the generation model generating compound molecules previously as a basis and use the molecular data evaluated by the experts above. The processor periodically fine-tunes the generation model so that the neural network model may recommend more expected compound molecules in subsequent generated compounds.

In step 460, it should be understood that the user may directly give instructions (actively) to the multimodal customized compound generation system to fine-tune each parameter of the neural network model. The multimodal customized compound generation system may also automatically (passively) fine-tune the generation model regularly (e.g., once a month). The multimodal customized compound generation system may use the fine-tuned neural network model for subsequent compound generation recommendations.

In the disclosure, the storage of the multimodal customized compound generation system further stores a plurality of modules, and the processor may execute a quality monitoring module in the plurality of modules to determine whether the information of the second compound meets the quality threshold value. That is, in step 460, the processor further displays the summary portion of the compound in a highlighted manner to show whether the molecular characteristics of the compound meet the quality threshold value.

In the disclosure, in order to reduce the time of compound generation recommendation and improve the quality of compounds recommended by the neural network model, the processor may also execute a performance monitoring module in the plurality of modules to monitor the training performance of the neural network model, thereby ensuring that the training results of the neural network model are directed toward quickly recommending high-quality compounds.

Based on the above, in the disclosure, compounds may be generated by taking into account the characteristics of target proteins in different dimensions via a multimodal compound generation system and method. The compounds have stronger binding characteristics with the target proteins than molecules produced by previous methods taking into account a single dimension. In addition, modified compounds may be produced from known compound molecules and used to train the neural network model to improve the quality of future compound production. The disclosure also provides a multimodal customized compound generation system for the user to input expected parameters to produce compounds that meet the user's expectations.

What is claimed is:

1. A multimodal compound generation system, comprising:
a storage storing a plurality of models; and
a processor coupled to the storage and configured to:
input a plurality of parameters to a neural network model in the models, wherein there is a first correlation between a first parameter and a second parameter in the parameters, and there is a second correlation between the second parameter and a third parameter in the parameters; and
obtain information of a first compound via the neural network model, wherein a first characteristic of the first compound is greater than a first threshold value, and a second characteristic of the first compound is greater than a second threshold value.

2. The multimodal compound generation system of claim 1, wherein the processor is further configured to:
execute a heterogeneous data fusion algorithm to pre-process the parameters to obtain a plurality of pre-processing parameters, wherein each of the pre-processing parameters has a same format.

3. The multimodal compound generation system of claim 2, wherein the parameters comprise a one-dimensional structure, a two-dimensional structure, and a three-dimensional structure.

4. The multimodal compound generation system of claim 1, wherein the processor is further configured to:
execute a feature extraction model in the models to extract a plurality of features of a target compound, wherein the features are related to the parameters.

5. The multimodal compound generation system of claim 1, wherein the storage further stores a plurality of modules, wherein the processor is further configured to:
execute a prediction module in the modules to predict an activity and a stability of the first compound.

6. The multimodal compound generation system of claim 5, wherein the processor is further configured to:
execute a training module in the modules to train the neural network model according to information of the first compound to obtain a trained neural network model; and
input the parameters into the trained neural network model to obtain information of a second compound.

7. The multimodal compound generation system of claim 5, wherein the processor is further configured to:
determine information of the first compound meets a first expectation.

8. The multimodal compound generation system of claim 1, wherein the first parameter is a polypeptide sequence, the second parameter is a secondary structure of a protein, and the third parameter is a three-dimensional structure of a protein.

9. A multimodal compound generation method, comprising:
inputting a plurality of parameters to a neural network model, wherein there is a first correlation between a first parameter and a second parameter in the parameters, and there is a second correlation between the second parameter and a third parameter in the parameters; and
obtaining information of a first compound via the neural network model, wherein a first characteristic of the first compound is greater than a first threshold value, and a second characteristic of the first compound is greater than a second threshold value.

10. The multimodal compound generation method of claim 9, further comprising:
executing a heterogeneous data fusion algorithm to pre-process the parameters to obtain a plurality of pre-processing parameters, wherein each of the pre-processing parameters has a same format.

11. The multimodal compound generation method of claim 10, wherein the parameters comprise a one-dimensional structure, a two-dimensional structure, and a three-dimensional structure.

12. The multimodal compound generation method of claim 9, further comprising:
executing a feature extraction model to extract a plurality of features of a target compound, wherein the features are related to the parameters.

13. The multimodal compound generation method of claim 9, further comprising:
executing a prediction module to predict an activity and a stability of the first compound.

14. The multimodal compound generation method of claim 13, further comprising:
executing a training module to train the neural network model according to information of the first compound to obtain a trained neural network model; and
inputting the parameters into the trained neural network model to obtain information of a second compound.

15. The multimodal compound generation method of claim 13, further comprising:
determining information of the first compound meets a first expectation.

16. The multimodal compound generation method of claim 9, wherein the first parameter is a polypeptide sequence, the second parameter is a secondary structure of a protein, and the third parameter is a three-dimensional structure of a protein.

17. A multimodal customized compound generation system, comprising:
a storage storing a plurality of models; and
a processor coupled to the storage and configured to:
obtain information of a first compound;
obtain a plurality of basic parameters according to the first compound; and
execute the method of claim 9 to obtain information of a second compound.

18. The multimodal customized compound generation system of claim 17, further comprising a user interface coupled to the processor, wherein the processor is further configured to:
obtain the first threshold value and the second threshold value via the user interface.

19. The multimodal customized compound generation system of claim 17, wherein the storage further stores a plurality of modules, wherein the processor is further configured to:
execute a quality monitoring module in the modules to determine whether the information of the second compound meets a quality threshold value.

20. The multimodal customized compound generation system of claim 19, wherein the processor is further configured to:
execute a performance monitoring module in the modules to monitor a training performance of the neural network model.
